(19) United States
(12) Patent Application Publication
(10) Pub. No.: US 2002/0169129 A1
(43) Pub. Date: Nov. 14, 2002

(54) Method for improving the quality of life of patients by administration of erythropoietin (RhuEPO)

(76) Inventor: Veronica C. Zaharia, New York, NY (US)

Correspondence Address: GOTTLIEB RACKMAN & REISMAN PC, 270 MADISON AVENUE, 8TH FLOOR, NEW YORK, NY, 100160601

(21) Appl. No.: 10/133,545

(22) Filed: Apr. 26, 2002

Related U.S. Application Data
(63) Continuation-in-part of application No. 09/872,630, filed on Jun. 1, 2001.
Application No. 09/872,630 is a division of application No. 09/335,076, filed on Jun. 17, 1999, now Pat. No. 6,274,158.
Application No. 09/335,076 is a continuation-in-part of application No. 09/018,815, filed on Feb. 4, 1998, now Pat. No. 5,951,996.
(60) Provisional application No. 60/091,598, filed on Jul. 2, 1998.
(60) Provisional application No. 60/125,253, filed on Mar. 19, 1999.
(60) Provisional application No. 60/287,206, filed on Apr. 28, 2001.

(51) Int. Cl. A61K 38/22

(52) U.S. Cl. 514/12

(57) ABSTRACT

A method for providing various benefits with the administration of different quantities of Erythropoietin. The method provides for enhancing the of quality of life by administration of Erythropoietin before a substantial increases in hemoglobin occurs. The improvement in quality of life is independent of the hemopoietic effect. In larger quantities the administration of RhuEPO leads to repair of vascular damage and leads to the redistribution of the iron trapped in storage organs, from where it cannot be used for red blood cell production, into the hemopoietic system leading to enhanced red blood cell production.

This application is a continuation-in-part of pending patent application Ser. No. 09/872,630, filed Jun. 1, 2001, which is a divisional application of patent application Ser. No. 09/335,076, filed Jun. 17, 1999, now U.S. Pat. No. 6,274,158, issued Aug. 14, 2001, which is a continuation-in-part of application Ser. No. 09/018,815, filed Feb. 3, 1998, now U.S. Pat. No. 5,951,996, issued Sep. 14, 1999 and provisional applications No. 60/091,598, filed Jul. 2, 1998 and No. 60/125,253, filed Mar. 19, 1999. This application also claims priority of provisional application No. 60/287,206, filed Apr. 28, 2001.

### FIELD OF THE INVENTION

This invention relates to treatments of patients by administration of erythropoietin (RhuEPO).

### BACKGROUND OF THE INVENTION

Erythropoietin is a glycoprotein which stimulates red blood cell production. It is produced naturally in the kidney and stimulates the division and differentiation of committed erythroid progenitors in the bone marrow. Recombinant Human Erythropoietin (RhuEPO) is an amino acid glycoprotein manufactured by recombinant DNA technology, has the same biological effects as endogenous erythropoietin. It is produced by mammalian cells into which the human erythropoietin gene has been introduced and contains the identical amino acid sequence of isolated natural erythropoietin.

Erythropoietin is primarily used to induce production of red blood cells to combat anemia, and not to stop diffuse bleeding. It is being used successfully in the treatment of anemia of chronic renal failure, anemia of cancer and in HIV patients. Erythropoietin is known to decrease the bleeding time in uremic (kidney failure) patients. In vitro and in vivo studies on uremic patients have shown an improved platelet endothelial cell interaction, which explains the shortening of the bleeding time, but there are no studies done of the hemostatic mechanism (clotting mechanism) induced by erythropoietin on non-uremic patients. There has been no recognition prior to the work of the present inventor of the significant limitation of chronic bleeding processes that can be achieved by the administration of erythropoietin in uremic or non-uremic patients.

It has been reported that the administration of Erythropoietin in quantities sufficient to substantially increase hemoglobin production results in the enhancement of quality of life of the patient.

### BRIEF DESCRIPTION OF THE PRESENT INVENTION

One aspect of the present invention is based upon the discovery that the enhancement of quality of life is not ties to substantial increases in hemoglobin but that it may result from lesser quantities of Erythropoietin than previously realized.

A. Contrary to the expectations of the prior art it has been discovered that patients free of anemia derive quality of life benefits from the administration of RhuEPO. The present invention is the administration of effective quantities of RhuEPO to such patients to bring about quality of life improvements(physical and mental) prior to any significant change in the Hemoglobin/Hematocrit.

B. In another aspect of the present invention, the administration of RhuEPO leads to repair of vascular damage.

C. In still another aspect of the present invention, RhuEPO leads to the redistribution of the iron trapped in storage organs, from where it cannot be used for red blood cell production, into the hemopoietic system leading to enhanced red blood cell production.

### DETAILED DESCRIPTION OF THE INVENTION

A. RhuEPO can improve the quality of life of patients. During RhuEPO treatment patients enjoy an improvement in their appetite and the patient's weight increases. During and following RhuEPO treatment a sense of physical and mental well being is reported by patients, which occurs prior to a significant increases in Hg (less than 1 g/dl).

During and after RhuEPO treatment the patients report better physical tolerance to daily life's demands which are not explainable by small (1 g/dl or less) increases of Hg. The patients report less shortness of breath, less fatigue and less palpitations. Their capacity to walk and work for longer periods of time improves.

Patients treated with RhuEPO report an improvement in their cognitive function. Patients who were previously depressed, lethargic, had a mild chronic state of mental confusion, impairment of their memory and delay in response to questions have a marked improvement during and following the treatment with RhuEPO. They have an improvement of their mood; clearing of their sensorium, they become more alert and a more rapid and appropriate response to questions is noticed. These improvements occur prior to significant changes in Hg (<1 mg/dl).

RhuEPO treatment induces excellent compliance with the treatment itself as a result of the fact that patients and their families are aware of the physical and psychological improvements induced by RhuEPO.

B. Treatment with RhuEPO has resulted in the shortening of the bleeding time in chronic renal failure patients on hemodialysis as well as in patients with normal kidney function, leading to better control of bleeding processes. RhuEPO administered topically on a limited part of a bleeding lesion, stops the bleeding in that area earlier than the natural cessation of bleeding in areas where no RhuEPO was applied.

During anticoagulation with Heparin, warfarin or aspirin patients can bleed from areas where there is preexisting vascular damage. RhuEPO can repair damaged vascular wall and lead to efficient and lasting hemostasis despite continuation of anticoagulation at therapeutic or several times the therapeutic range for anticoagulation.

RhuEPO can be used in diseases where diffuse vascular damage is the main problem in order to repair such damage Examples of such diseases are: vasculitis (autoimmune), capillary leak syndrome, diabetic, arteriosclerotic or other damage to vessels leading to overt bleeding or oozing of blood cerebral bleeding, aneurysms, damage to blood vessels caused by traumatic, chemical or physical agents or postoperatively.

In TTP (thrombotic thrombocytopenic purpura), HUS (hemolytic uremic syndrome) where extensive endothelial damage is suspected RhuEPO can be used to improve the endothelial damage.

RhuEPO works at the platelet subendothelial level where it promotes primary plug formation and repair of damaged vessels. It is possible that it also decreases the protein C, protein S and Antithrombin III level.

In patients with vascular damage due to vesicant drugs (such as Adriamycin, Vincristin), with catastrophic extravasation of the vesicant drug and ongoing chemical burn, RhuEPO could enhance the repair of the vascular wall and thus limit the damage which at the present time can only be done by surgical excision of the affected area.

C. Correction of anemia of iron deficiency in chronically bleeding patients by RhuEPO. RhuEPO can control transfusion dependent bleeding. Iron loss is prevented and increased amounts of iron are incorporated into red cells leading to an increased red blood cell hemoglobinisation. This is expressed in an increase in MCH (mean corpuscular hemoglobin). Three severely anemic transfusion dependent bleeding patients were studied.

Patient # 1 was on Aspirin, Heparin and Coumadin for an acute myocardial infarct. She was difficult to control and developed PT&PTT often several times the normal value. An enlarging massive RPN (retroperitoneal) bleeding developed compromising the kidney function. After 5 Units (U) packed red blood cells (PRBC), 9U fresh frozen plasma and Vitamin K injections the bleeding continued. Following RhuEPO injections the bleeding stopped, never to recur.

Patient # 2 bled due to radiation (Rx) proctitis; 20 U PRBC/6 months were transfused. Local hemostatic measures failed. RhuEPO administration led to hemostasis, and its discontinuation was followed by rebleeding. Restarting RhuEPO led to control of bleeding.

Patient # 3 bled for 2 years due to angiodysplasia of colon requiring PRBC transfusion every 2 months despite repeated local hemostatic attempts. Following RhuEPO administration the bleeding stopped.

All patients became transfusion independent following RhuEPO administration.

| | | increase | increase in | RhuEPO | |
|---|---|---|---|---|---|
| | | in | MCH (pg) / | dose (U / kg | Side |
| Nr | Bleeding lesion | Hg(g/dl) / | time (days) | / week) | effects |
| 1 | RPN bleeding | 3.7/20 | 2.1/20 | 512 | 0 |
| 2 | Rx. proctitis | 3.0/26 | 2.2/26 | 235 | 0 |
| 3 | angiodysplasia | 0.2/28 | 3.1/28 | 182 | 0 |

RhuEPO has been shown to improve platelet/subendothelial interaction and to decrease proteins C, S and ATIII. In a bleeding patient RhuEPO:

enhances hemostasis.

boosts hemopoiesis.

raises the MCH.

This is achieved by preventing RBC and hence iron loss and directly incorporating the saved iron into the erythroid precursors, resulting in increased red blood cell hemoglobin content (MCH). Overall in a bleeding patient RhuEPO shifts a negative iron balance to a positive one.

RhuEPO can shift iron trapped in the reticuloendothelial system of patients with anemia of chronic disease into the erythropoietic cells, leading to an increased hemoglobinisation of the red blood cells. As a result of this effect the level of the blood Ferritin goes down and the Hemoglobin and MCH increase. These effects combined lead to a major improvement in the quality of life of patients with anemia of chronic disease.

In some of the chronically anemic bleeding patients treated with RhuEPO with subsequent control of the bleeding process, no reoccurrence of the bleeding process occurs for weeks, months or even years after the treatment with RhuEPO was discontinued. The hemostatic effect of RhuEPO is sometimes a lasting one.

RhuEPO can be used in Hemochromatosis or other iron overload conditions such as congenital hemolytic anemias (such as thalassemia) to unload the iron trapped in nonhemic cells (non-erythropoietic), and possibly channel it into hemopoietic cells.

Exogenously administered RhuEPO works in anemia of chronic disease patients even in the presence of normal endogenous levels of Erythropoietin possibly due to a nonfunctioning endogenous Erythropoietin (as far as erythropoiesis or switching the iron from the RES into the hematopoietic cell line or hemostasis is concerned).

The treatment with RhuEPO of patients with anemia of chronic disease, anemia of cancer or due to chemotherapy or due to recurrent bleeding leads to a marked improvement in the quality of life of these patients which appears to be partly secondary to the elevation of Hg and partly independent of it.

RhuEPO appears to be a crucial factor in the proper distribution of iron in the body. In the anemia of chronic disease, RhuEPO leads to an improvement of Hg, which in turn leads to an improvement of the chronic disease due to a better tissue oxygenation.

In congenital hemolytic anemias RhuEPO not only;

stimulates the production of new RBC's it also

removes the excessive iron accumulated in the body and

channels it into the rapidly produced new RBC's.

Through all these mechanisms, RhuEPO leads to an increase in Hg and improvement of the quality of life of patients.

1. A method comprising administration of RhuEPO to a patient an effective dosage to increase the quality of life of the patient prior to an increase in Hg of 1 g/dl.

2. The method of claim 1, wherein the increase in the quality of life comprises an improvement in appetite.

3. The method of claim 1, wherein the increase in the quality of life comprises an increase in the patient's weight.

4. The method of claim 1, wherein the increase in the quality of life comprises a sense of physical and mental well being.

5. The method of claim 1, wherein the increase in the quality of life comprises better physical tolerance to daily life's demands.

6. The method of claim 1, wherein the increase in the quality of life comprises less shortness of breath, less fatigue and less palpitations.

7. The method of claim 1, wherein the increase in the quality of life comprises increased capacity to walk and work for longer periods of time.

8. The method of claim 1, wherein the increase in the quality of life comprises improvement in their cognitive function.

9. The method of claim 1, wherein the increase in the quality of life comprises improvement of mood, clearing of sensorium, becoming more alert and a more rapid and appropriate response to questions.

10. The method of claim 1, wherein RhuEPO is administered to patients with Alzheimer's disease in order to achieve improvements in cognitive functions regardless of their Hg level.

11. The method comprising administration of RhuEPO to a patient with vascular damage (s.a. vasculitis capillary leak syndrom, aneurysms, chemical or physical damage to vessel) in TTP or HUS can lead to repair of damaged vessel

12. The method comprising administration of RhuEPO topically to patients with superficial bleeding lesions with enhancement of the hemostatic process.

13. A method for limiting chronic blood loss, comprising administering an effective quantity of RhuEPO to prevent iron loss, and to channel the saved iron directly into the erythroid precursors leading to increased hemoglobin level, increased MCH, and increased RBC hemoglobinisation.

14. The method of claim 13, wherein iron trapped in the reticuloendothelial system in patients with anemia of chronic disease or hematologic malignancy is made available to erythropoiesis during RhuEPO treatment.

15. The method of claim 13, wherein there results a shortening of the bleeding time in chronic renal failure patients on hemodialysis as well as in patients with normal kidney function, leading to better control of bleeding processes.

16. The method of claim 13, wherein RhuEPO is administered topically on a limited part of a bleeding lesion and results in the bleeding in that area stopping earlier than the natural cessation of bleeding in areas where no RhuEPO was applied.

17. The method of claim 13, wherein RhuEPO is used in iron overload conditions to unload the iron trapped in nonhemic cells (non-erythropoietic).

18. The method of claim 13, wherein exogenously administering RhuEPO in anemia of chronic disease patients having normal endogenous levels of Erythropoietin.

19. The method of claim 1, wherein RhuEPO is administered during anticoagulation with Heparin, warfarin or aspirin to repair damaged vascular wall and lead to efficient and lasting hemostasis despite continuation of anticoagulation at therapeutic or several times the therapeutic range for anticoagulation.

20. The method of claim 19, wherein RhuEPO works at the platelet subendothelial level to promote primary plug formation and repair of damaged vessels.

21. The method of claim 19, wherein the RhuEPO decreases the protein C, protein S and Antithrombin III level.

22. A method comprising administration of RhuEPO to a patient with vascular damage due to vesicant drugs (such as Adriamycin, Vincristin), with catastrophic extravasation of the vesicant drug and ongoing chemical burn, an effective dosage of RhuEPO to enhance the repair of the vascular wall and thus limit damage thereto.

23. A method comprising administration of RhuEPO to a patient with congenital hemolytic anemias RhuEPO to stimulate the production of new RBC's, remove excessive iron accumulated in the body and channels the excess iron said new RBC'S.
